(19) United States
(12) Patent Application Publication
(10) Pub. No.: US 2024/0269366 A1
(43) Pub. Date: Aug. 15, 2024

(54) Drainage System Including Actuation Device For Providing A Negative Pressure For Draining And Collecting Bodily Fluids

(71) Applicant: Becton Dickinson and Company, Franklin Lakes, NJ (US)

(72) Inventors: Michael Plishka, Lake Villa, IL (US); Katherine Konsor, Deer Park, IL (US); Erin L. Schaus, Elk Grove Village, IL (US); Amy A Mason, Vernon Hills, IL (US)

(73) Assignee: Becton Dickinson and Company, Franklin Lakes, NJ (US)

(21) Appl. No.: 18/568,646

(22) PCT Filed: Jun. 11, 2021
(86) PCT No.: PCT/US2021/037045

§ 371 Date: Dec. 8, 2023

(51) Int. Cl. A61M 1/00

(52) U.S. Cl. CPC A61M 1/66; A61M 1/78; A61M 2207/00

(57) ABSTRACT

A drainage system for draining bodily fluid includes a fluid-receiving body having a fluid-receiving chamber located therein that receives a bodily fluid through an inlet. An actuation device includes a housing with a container-receiving chamber that is sized and configured to receive a pre-evacuated container therein. The actuation device includes an actuation member that, upon actuation, moves either the pre-evacuated container or a piercing element toward the other of the pre-evacuated container or piercing element such that the piercing element pierces a seal of the pre-evacuated container providing communication of a negative pressure inside the pre-evacuated container to a fluid-receiving passageway to draw bodily fluid into the fluid-receiving passageway and toward the fluid-receiving body.

### TECHNICAL FIELD

The present specification generally relates to drainage systems and methods for draining bodily fluid from body cavities and, more specifically, to drainage systems and methods including fluid collection apparatuses for draining and collecting bodily fluids.

### BACKGROUND

The pleural cavity is the thin, fluid-filled space between the two pulmonary pleurae (known as visceral and parietal) of each lung. A pleura is a serous membrane that folds back onto itself to form a two-layered membranous pleural sac. The pleural space is normally filled with approximately five to 20 mL of serous fluid. The turnover of fluid in the pleural space is normally quite rapid—roughly 35 to 75% of the total fluid per hour, so that 5 to 10 L of fluid moves through the pleural space each day. A pleural effusion is a build-up of fluid in the pleural space. A pleural effusion may also be referred to as effusion or pulmonary effusion. The type of fluid that forms a pleural effusion may be categorized as transudate or exudate.

Peritoneal effusion or ascites refers to an excess collection of fluid in the abdominal cavity, such as more than 25 mL of fluid. The most common cause of such excess fluid collection is liver cirrhosis and other causes include cancer and pancreatitis.

There are a number of drainage systems for managing recurrent pleural effusions and ascites at home. Two such systems are the PleurX™ and PeritX™ drainage systems, commercially available from Becton, Dickinson and Company. These systems utilize a catheter and drainage bottles that collect fluid. The catheter is inserted, typically as a simple outpatient procedure, in the chest for draining pleural effusions or in the abdomen for ascites. An end of the catheter stays outside of the body, hidden under a bandage when not in use. To drain, the end of the catheter is connected to a drainage line on the bottle that is pre-evacuated to provide a negative pressure, and the negative pressure inside the bottle is used to draw out the bodily fluid.

### SUMMARY

According to a first embodiment, a drainage system for draining bodily fluid includes a fluid-receiving body having a fluid-receiving chamber located therein that receives a bodily fluid through an inlet. An actuation device includes a housing with a container-receiving chamber that is sized and configured to receive a pre-evacuated container therein. The actuation device includes an actuation member that, upon actuation, reduces a distance between a seal of the pre-evacuated container and a piercing element such that the piercing element pierces the seal of the pre-evacuated container providing communication of a negative pressure inside the pre-evacuated container to a fluid-receiving passageway to draw bodily fluid into the fluid-receiving passageway and toward the fluid-receiving body.

In another embodiment, a method of forming a drainage system for draining a bodily fluid is provided. The method includes connecting a first drainage tube to an inlet of a housing of an actuation device. The actuation device includes the housing including a container-receiving chamber that is sized and configured to receive a pre-evacuated container therein. The actuation device includes an actuation member that, upon actuation, reduces a distance between a seal of the pre-evacuated container and a piercing element such that the piercing element pierces a seal of the pre-evacuated container providing communication of a negative pressure inside the pre-evacuated container to a fluid-receiving passageway to draw bodily fluid into the fluid-receiving passageway and toward the fluid-receiving body. A pre-evacuated container is placed in the container receiving chamber.

In another embodiment, a drainage system for draining bodily fluid includes a fluid-receiving body having a fluid-receiving chamber located therein that receives a bodily fluid through an inlet. An actuation device includes a housing with a container-receiving chamber sized and configured to receive a pre-evacuated container therein. The actuation device includes an actuation device comprising a housing with a container-receiving chamber sized and configured to receive a pre-evacuated container therein. The actuation device including an actuation member that, upon actuation, reduces a distance between a seal of the pre-evacuated container and a piercing element such that the piercing element pierces a seal of the pre-evacuated container. Bodily fluid bypasses the pre-evacuated container through a fluid-receiving passageway and is received by the fluid-receiving body.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a drainage system including an actuation device with a pre-evacuated container housed therein, according to one or more embodiments shown and described herein;

FIG. 2 is a diagrammatic side view of the actuation device of FIG. 1 in isolation, according to one or more embodiments shown and described herein;

FIG. 3 is an exploded view of the actuation device of FIG. 2, according to one or more embodiments shown and described herein;

FIG. 4A is a diagrammatic section view of the actuation device of FIG. 3 in an assembled configuration, according to one or more embodiments shown and described herein;

FIG. 4B is a detail view of a flow control device of the actuation device of FIG. 4A, according to one or more embodiments shown and described herein;

FIG. 4C shows the flow control device of FIG. 4B in operation, according to one or more embodiments shown and described herein;

FIG. 4D illustrates a detail section view of a portion of another actuation device showing an end cap, according to one or more embodiments shown and described herein;

FIG. 4E illustrates another actuation device including a clamping member, according to one or more embodiments shown and described herein;

FIG. 5 illustrates a method of using the drainage system of FIG. 1 including the actuation device, according to one or more embodiments shown and described herein;

FIG. 6 illustrates another embodiment of an actuation device of a drainage system, according to one or more embodiments shown and described herein;

FIG. 7 illustrates another embodiment of an actuation device of a drainage system, according to one or more embodiments shown and described herein;

FIG. 8 illustrates another embodiment of an actuation device of a drainage system, according to one or more embodiments shown and described herein;

FIG. 9 illustrates another embodiment of an actuation device of a drainage system, according to one or more embodiments shown and described herein;

FIG. 10 illustrates another embodiment of an actuation device of a drainage system, according to one or more embodiments shown and described herein;

FIG. 11 illustrates another embodiment of an actuation device of a drainage system, according to one or more embodiments shown and described herein;

FIG. 12 illustrates another embodiment of an actuation device of a drainage system, according to one or more embodiments shown and described herein;

FIG. 13 illustrates another embodiment of an actuation device of a drainage system, according to one or more embodiments shown and described herein; and

FIG. 14 illustrates another embodiment of an actuation device of a drainage system, according to one or more embodiments shown and described herein.

### DETAILED DESCRIPTION

Embodiments described herein are generally directed to drainage systems for draining bodily fluids. The drainage systems may include a fluid-receiving body having a fluid-receiving chamber located therein that receives a bodily fluid through an inlet. An actuation device includes a housing with a container-receiving chamber that is sized and configured to receive a pre-evacuated container therein. The actuation device includes an actuation member that, upon actuation, moves either the pre-evacuated container or a piercing element toward the other end of the pre-evacuated container or piercing element thereby reducing a distance between a seal of the pre-evacuated container and the piercing element. The piercing element pierces the seal of the pre-evacuated container providing communication of a negative pressure inside the pre-evacuated container to a fluid-receiving passageway that extends through the housing. The negative pressure is used to draw bodily fluid out of a body cavity or other location toward the fluid-receiving body to initiate drainage such that a siphon effect can continue drainage.

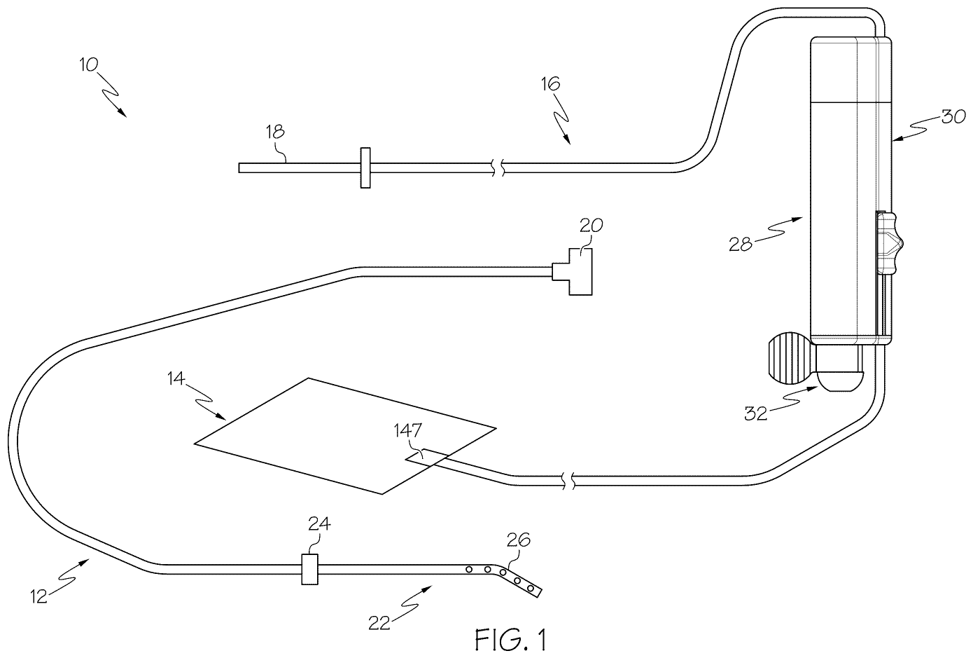

Referring to FIG. 1, a drainage system 10 for use in draining bodily fluids from body cavities includes a conduit 12 that may be partially implanted in a patient's body and a fluid-receiving body 14 that can be fluidly connected to the conduit 12. The fluid-receiving body 14 may be connected to the conduit 12 by a drainage line 16 that extends from the fluid-receiving body 14 to the conduit 12. In some embodiments, the drainage line 16 may include a connector 18 that connects to a one-way valve 20. As an example, the one-way valve 20 may be a two-piece valve that is normally closed, but that opens with insertion of the connector 18 to allow bodily fluid to flow through the drainage line 16. A distal end portion 22 of the conduit 12 may be implanted in the patient's body for pleural or peritoneal drainage or other body location for draining a bodily fluid. In some embodiments, the distal end portion 22 may include a sealing cuff 24 and fenestrations 26 through which the bodily fluid may enter the conduit 12 and be carried from the pleural, peritoneal or other cavity space.

An actuation device 28 is used to supply negative pressure through the drainage line 16 and the conduit 12 thereby providing a suction to automatically draw bodily fluid from the cavity in which the conduit 12 is inserted toward the fluid-receiving body 14. As used herein, the terms “negative pressure,” “suction,” vacuum” and “partial vacuum” may be used interchangeably and refer to a force over an area produced by a difference in pressure. Pressure may be referred to as “negative” in reference to atmospheric pressure (i.e., gauge pressure). It should be noted that while using the actuation device for draining bodily fluids from body cavities is described primarily herein, the actuation devices may be used to drain bodily fluids from other locations, such as wounds. The actuation device 28 includes an outer housing 30 and an actuation member 32 that moves relative to the outer housing 30 and that is used to generate a negative pressure differential relative to pressure within the body cavity to initiate fluid drainage from the body cavity. Because the drainage line 16 passes through the outer housing 16, the actuation device 28 may be considered in-line with the drainage line 16. In other embodiments, the actuation devices may be out-of-line (see FIGS. 8-12). While not drawn to scale, the actuation device 28 is sized to fit within a patient's hand (see FIG. 11). As will be described in greater detail below, movement of the actuation member 32 relative to the outer housing 30 moves a piecing element toward a seal of a pre-evacuated container in order to pierce the seal and supply negative pressure to the drainage line 16. As used herein, the term “housing” is meant broadly to include any protector for the pre-evacuated container and may be closed or only partially closed and may or may not encase the entire pre-evacuated container.

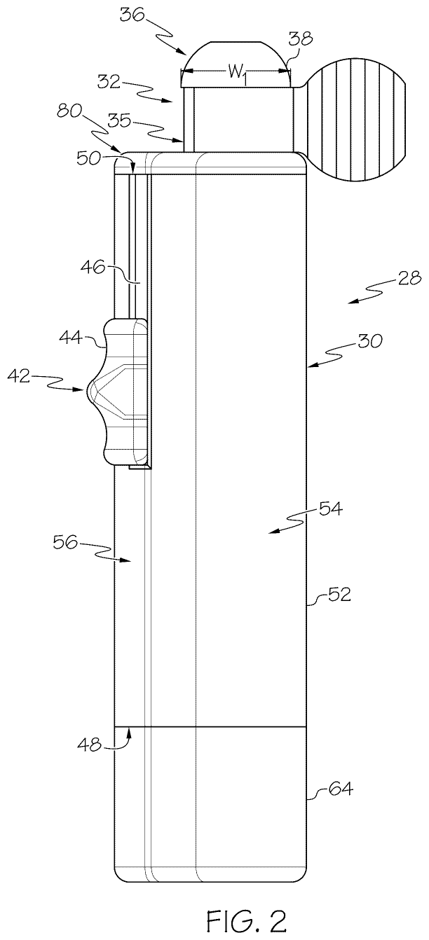

FIG. 2 illustrates a more detailed, side view of the actuation device 28. The actuation device 28 includes the outer housing 30 and the actuation member 32. In this example, the actuation member 32 includes an actuation portion 36 and a sleeve body 35. The actuation portion 36 is located at an end 38 of the sleeve body 35 and has a maximum width W1 that is greater than the end 38 of the sleeve body 35 such that the actuation portion 36 overhangs an interlocking member 80 that is used to slidably connect the actuation member 32 to the outer housing 30. A flow adjustment member 42 is movably connected to the outer housing 30. The flow adjustment member 42 includes a slide 44 that is slidably received within one or more slots 46 formed in the outer housing 30. The slide 44 can be moved along a length of the outer housing 30 in order to increase and decrease a flow rate of the bodily fluid through the drainage line 16.

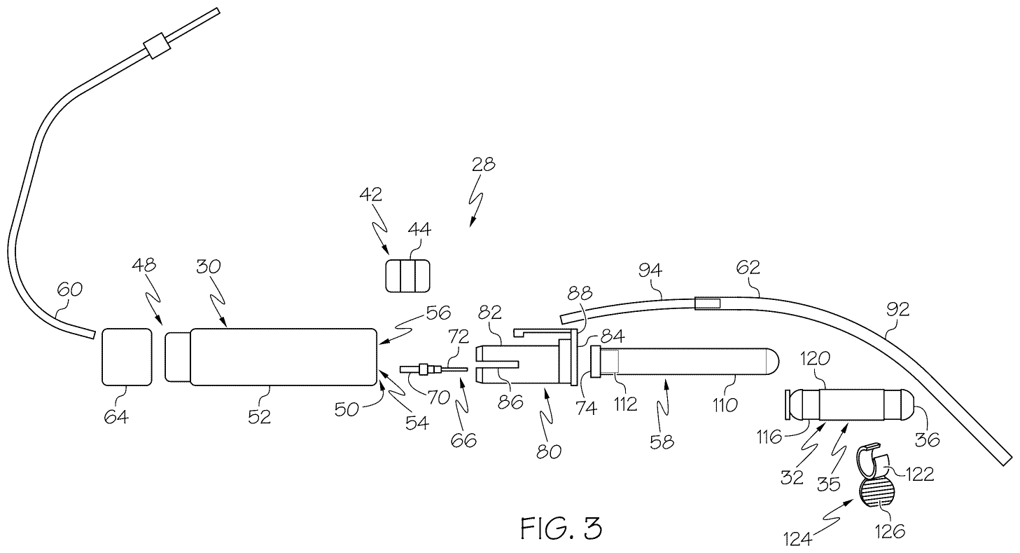

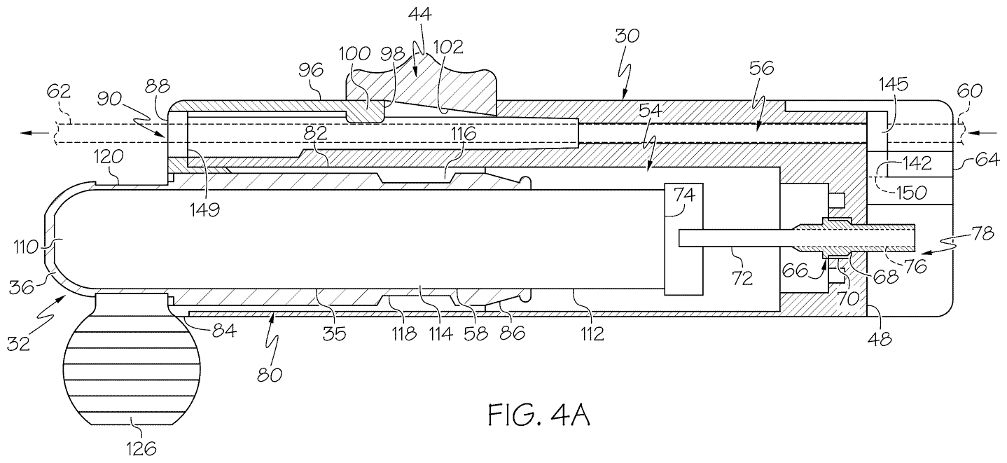

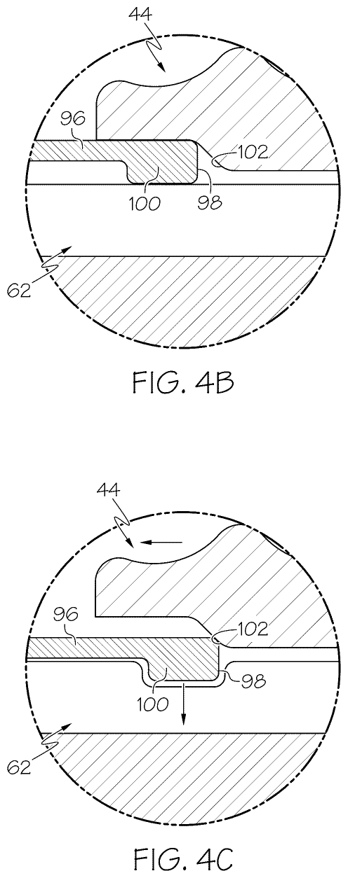

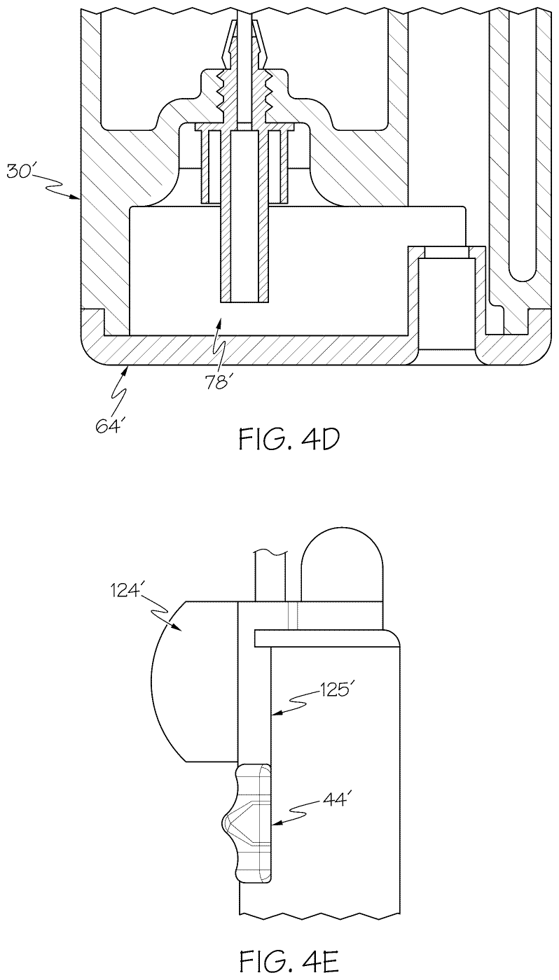

Referring to FIG. 3, an exploded view of the actuation device 28 is illustrated. The actuation device 28 includes the outer housing 30, actuation member 32 and flow adjustment assembly 42. The outer housing 30 includes opposite ends 48 and 50 and a housing body 52 that forms both a container-receiving chamber 54 and a fluid-receiving passageway 56 inside the housing body 52 (FIG. 4A). The container-receiving chamber 54 is sized and configured to receive a pre-evacuated container 58 therein and the fluid-receiving passageway 56 is sized and configured to connect to first and second drainage tubes 60 and 62. The first and second drainage tubes 60 and 62 and the fluid-receiving passageway 56 may together form the drainage line 16 (FIG. 1). An end cap 64 may be used to cover the end 48 of the outer housing 30. The end cap 64 may also provide communication between the pre-evacuated container 58 and the fluid-receiving passageway 56, which will be described in greater detail below. In some embodiments, the end cap 64 may be formed as a monolithic part of the outer housing 30. However, use of the removable end cap 64 can facilitate placement of a piercing device 66 within the container-receiving chamber 54 by exposing a mounting opening 68 (FIG. 4A) in the end 48 of the outer housing 30 and then providing an air-tight seal around the mounting opening 68.

Referring to FIG. 4A, the mounting opening 68 is sized and shaped to receive a lock portion 70 of the piercing device 66. As one example, the lock portion 70 may be a luer-type lock that can be pulled through the mounting opening 68 and then turned to lock the lock portion 70 in place within the mounting opening 68. A narrow tube portion 72 may serve as a piercing element that can pierce a seal 74 that covers the pre-evacuated container 58. A bore 76 may extend all the way through the piercing device 66 to provide communication between the pre-evacuated container 58 and a vacuum chamber 78 that is formed between the end cap 64 and the end 48 of the outer housing 30. In the example of FIG. 4D, another embodiment of an end cap 64′ is relatively thin compared to end cap 64 and covers and closes a vacuum chamber 78′ that is formed within outer housing 30′.

Referring now to FIGS. 3 and 4A, the interlocking member 80 may connect the second drainage tube 62 and the actuation member 32 to the outer housing 30. The interlocking member 80 may include an insertion body 82 that is sized to be received within the container-receiving chamber 54. The insertion body 82 may be sized to frictionally or otherwise engage the housing body 52. An end flange 84 extends outward from the insertion body 82 that is sized to engage the end 50 of the outer housing 30. In some embodiments, the insertion body 82 may include deflectable catch members 86 that can deflect during insertion of the interlocking member 80 into the container-receiving chamber 54 and the snap back into their original positions to lock the insertion body 82 within the container-receiving chamber 54.

The interlocking member 80 positions the second drainage tube 62 within the outer housing 30. The interlocking member 80 includes a tube-receiving flange 88 that has a tube-receiving opening 90 (FIG. 4A) through which the second drainage tube 62 is threaded. In the illustrated example, the second drainage tube 62 includes a larger outer diameter portion 92 and a smaller outer diameter portion 94. The larger and smaller outer diameter portions 92 and 94 may be connected together or they may formed together as a single, monolithic tube. Both the larger outer diameter portion 92 and the smaller outer diameter portion 94 may be threaded through the tube-receiving opening 90. The interlocking member 80 further includes a resiliently-cantilevered flow control arm 96 that extends outward from the tube-receiving flange 88 alongside the second drainage tube 62. The flow control arm 96 extends from the tube-receiving flange 88 to a free end 98 that includes an enlarged portion 100. It should be noted that while a flow control arm 96 is illustrated as an example, clamps and other suitable devices may be used to control fluid flow rate. For example, a flow control clamp may be located along the second drainage tube 62 upstream of the actuation device 28.

Referring to FIGS. 4B and 4C, the slide 44 includes an inner surface 102 that is tapered inwardly toward the second drainage tube 62. The inner surface 102 can act as a cam surface that can be used to deflect the free end 98 and the enlarged portion 100 toward the second drainage tube 62 a desired amount in order to reduce a size of a lumen that extends therethrough. In particular, linear movement of the slide 44 along the slot 46 (FIG. 2) causes the free end 98 of the flow control arm 96 to travel along the inner surface 102 either toward the second drainage tube 62 or away from the second drainage tube 62 depending on the direction of travel of the slide 44. In the illustrated embodiment, movement of the slide 44 toward the tube-receiving opening 90 reduces or can even close the lumen of the second drainage tube 62 thereby reducing or even blocking flow of bodily fluid from the body cavity toward the fluid-receiving body 14 (FIG. 1). Movement of the slide 44 away from the tube-receiving opening 90 increases or fully opens the lumen of the second drainage tube 62 thereby increasing the flow of bodily fluid from the body cavity toward the fluid-receiving body 14. The slide 44 may move freely and be held in place manually or a latching device (such as a ratchet) may be provided that can be used to lock the slide 44 in a desired position relative to the outer housing 30 or both may be used (FIG. 4A). For example, the slide 44 may be moved freely until a latching device is used to lock the slide 44 in a desired position.

The actuation member 32 may include the sleeve body 35 and the actuation portion 36 that closes an end of the sleeve body 35. The sleeve body 35 may define a volume that is sized to receive a closed end 110 of the pre-evacuated container 58 therein and engage the closed end 110 within the actuation portion 36 (FIG. 4A). The length of the sleeve body 35 may be less than the length of the pre-evacuated container 58 such that, when the pre-evacuated container is fully inserted in the sleeve body 35, a sealed end 112 of the pre-evacuated container 58 protrudes outwardly beyond the sleeve body 35 (FIG. 4A). In some embodiments, one or more openings may be provided through the sleeve body 35 so as to allow air out of the interior of the sleeve body to facilitate manufacture. One or more openings through the sleeve body 35 may facilitate removal and replacement of the pre-evacuated container from the actuation device 28, for example if desired.

As shown by FIG. 4A, the actuation member 32, with the pre-evacuated container 58 inserted in the sleeve body 35 and pressed against the actuation portion 36, extends through a container-receiving opening 114 of the interlocking member 80 and is located in the container-receiving chamber 54. The sleeve body 35 may include one or more detent notch 116 that is sized to receive an inwardly extending detent 118 or projection that extends radially inward within the insertion body 82 of the interlocking member 80. The detent 118 engages the sleeve body 35 within the detent notch 116 and inhibits movement of the sleeve body 35 out of the container-receiving chamber 54. It should be noted that any of the end cap 62, interlocking member 80, actuation member 32 and first and second drainage tubes 60 and 62 may be held in place on or within the outer housing 30 by any suitable method, such as adhesive, snap fit, welding or combinations of any suitable methods.

Referring again to FIGS. 3 and 4A, the sleeve body 35 may include another notch 120 that is located between the actuation portion 36 and the rest of the sleeve body 35. The notch 120 is sized to receive a clamp 122 of a clamping member 124. Referring particularly to FIG. 4A, the clamping member 124 includes an enlarged tab 126 that can provide increased area for manual grasping by a patient. The clamp 122 clamps around the sleeve body 35 and provides a barrier between the actuation portion 36 and the outer housing 30 to inhibit depressing the actuation portion 36 relative to the outer housing 30 and moving the pre-evacuated container 58 toward the piercing device 66 unintentionally. In some embodiments, as shown by FIG. 4E, a clamping member 124′ may also inhibit sliding movement of slide 44′ until the clamping member 124′ is removed, for example, using a blocking element 125′. This can inhibit unintended closing or restriction of the tube 62 during shipping and reduce remedial actions taken by a patient who may have limited dexterity.

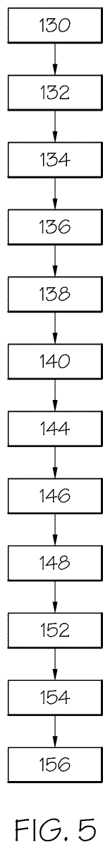

Referring to FIG. 5, in operation, the connector 18 of the drainage line 16 is inserted into the one-way valve 20 of the conduit 12. The connector simultaneously opens the one-way valve 20 as the connector 18 is inserted therein and provides communication between the fluid-receiving body 14 and a body cavity (FIG. 1) at step 130. At step 132, the fluid-receiving body 14 may be positioned on a surface or hung in a position that is below the body cavity. As an example, the patient may be seated and the fluid-receiving body 14 may be placed on the floor. At step 134, the enlarged tab 126 of the clamping member 124 is grasped manually and then pulled with a force sufficient to remove the clamp 122 from around the sleeve body 35. Once removed, the actuation portion 36 can be pressed manually and moved relative to the outer housing 30 at step 136. In some embodiments, the slider 44 may not be provided and a flow control device that is separate from the outer housing 30 may be provided.

Referring also to FIG. 4A, movement of the actuation portion 36 inward toward the outer housing 30 causes the pre-evacuated container 58 to move toward the piercing device 66 at step 138. The piercing device 66 has the bore 76 that extends therethrough such that negative pressure within the pre-evacuated container 58 can be communicated to the vacuum chamber 78 (FIG. 4A) that is provided between the end cap 64 and the outer housing 30. The actuation portion 36 can be depressed a sufficient amount toward the outer housing 30 such that the piercing element 72 pierces the seal 74 thereby generating the partial vacuum into the vacuum chamber 78 at step 140. The vacuum chamber 78 is in communication with the fluid-receiving passageway 56 which is, in turn, in communication with both the first and second drainage tubes 60 and 62 though a vacuum passageway 142 extending between the fluid-receiving passageway 56 and the vacuum chamber 78. The vacuum passageway 142 is located at a gap 145 that is provided between ends of the first drainage tube 60 and the second drainage tube 62. The gap 145 is provided so that the negative pressure can be introduced to the drainage line 16. In some embodiments, the fluid-receiving body 14 includes a check valve 147 (FIG. 1) that only allows fluid flow toward the fluid-receiving body 14. Fluids may then be drawn from the body cavity and into the drainage line 16. This check valve 147 can be placed anywhere within the system along the drainage tube 62. The closer the check valve 147 is to the actuation device 28, the more the negative pressure that may be experienced at the body cavity and more quickly. When the partial vacuum is created in the vacuum chamber 78, the pressure in the fluid-receiving passageway 56 is reduced which is communicated through the first and second tubes 60 and 62 and through the entire drainage line 16 at step 144. Because the fluid-receiving body 14 is already deflated and/or use of one-way check valve 147, the negative pressure is communicated primarily to the body cavity through the drainage line 16 at step 146, which can be used to draw bodily fluid from the body cavity and into the drainage line 16. In some embodiments, a check valve may be placed upstream of the actuation device 28 along the first drainage tube 60, which can prevent fluids from traveling back toward the body cavity.

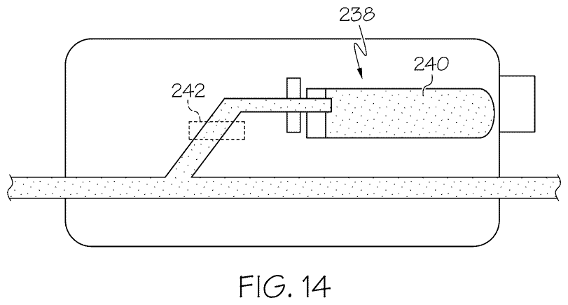

Fluids including bodily liquids and gases can be drawn from the body cavity and into the drainage line 16 at step 148 using the negative pressure that is communicated from the pre-evacuated container 58. The bodily fluids enter the fluid-receiving passageway 56 through a fluid inlet 149. A one-way valve, represented by element 150, may be located at or in the vacuum chamber 78 to prevent the bodily fluids from passing from the fluid-receiving passageway 56 into the vacuum chamber 78 thereby bypassing the pre-evacuated container 58 at step 152. In this regard, the pre-evacuated container 58 may be liquidly isolated from the fluid-receiving passageway 56 and the bodily fluid is directed into the first drainage tube 60 and then into the fluid-receiving body 14. In other embodiments, a one-way valve may not be used and the pre-evacuated container 58 may fill with bodily fluid while bodily fluid also bypasses the pre-evacuated container 58 (see FIG. 14). Once the drainage begins using the negative pressure provided by the pre-evacuated container 58, the drainage of the bodily fluid from the body cavity may continue as a siphon under the influence of gravity alone at step 154. At step 156, the flow rate of the bodily fluid from the body cavity can be controlled (increased or decreased) using the slider 44 as discussed above.

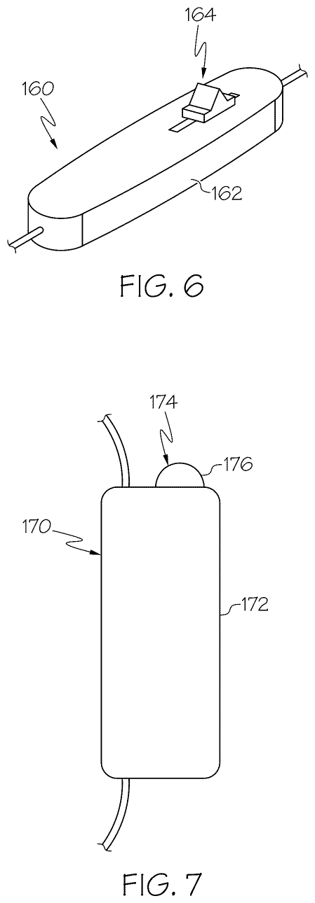

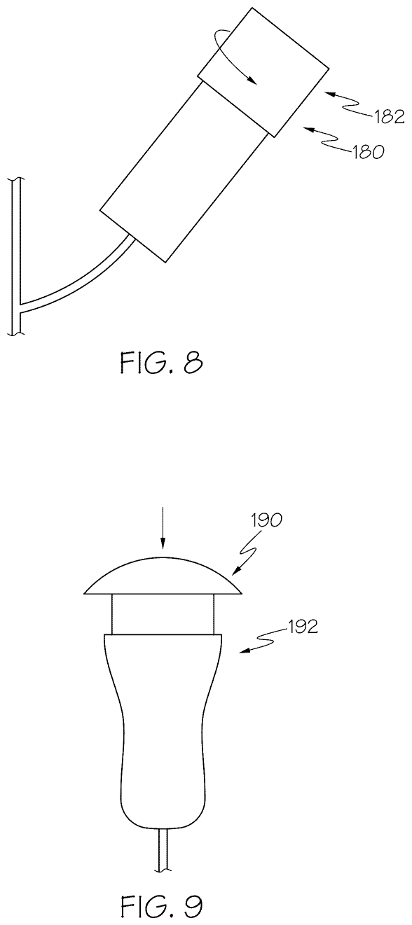

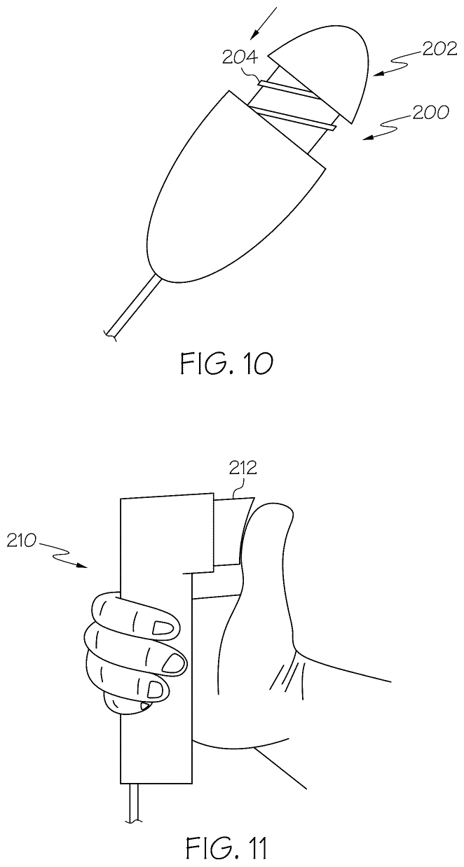

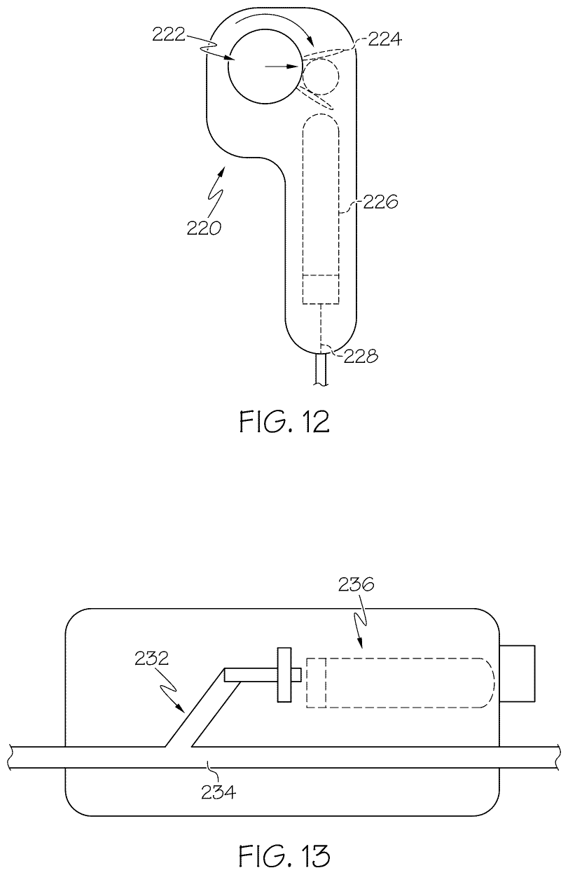

While a button-type actuation member is described above, other actuation member types can be used. For example, referring to FIG. 6, another actuation device 160 may include similar components as those described above including an outer housing 162, pre-evacuated container and fluid-receiving passageway. In this embodiment, a thumb-actuated slider 164 may be used to move a pre-evacuated container toward the piercing element. Referring to FIG. 7, in another embodiment, an actuation device 170 includes an outer housing 172 that exposes an end portion 174 of a pre-evacuated container 176. In this embodiment, the end portion 174, itself, is the actuation member and can be depressed. Referring to FIG. 8, another embodiment of an actuation device 180 includes a rotational actuation member 182 that can be used to move a pre-evacuated container toward a piercing element. Any suitable device can be used the converts the rotational motion of the actuation member 182 into linear motion, such as threads, gears, etc. Referring to FIG. 9, in another embodiment, an actuation device 190 includes an enlarged actuation member 192 that can easily be palm-actuated. In another embodiment, referring to FIG. 10, an actuation device 200 includes an actuation member 202 that both rotates and moves linearly using threads 204 to move a pre-evacuated container. Referring to FIG. 11, an actuation device 210 is provided that includes a side actuation member 212 in the form of a push button. For example, a cam arrangement (e.g., similar to slider 44) can be used to move a pre-evacuated container toward a piercing element. Referring to FIG. 12, another actuation device 220 is illustrated that includes an actuation member 222 in the form of a rotating side knob. Paddles or projections 224 can be used to move a pre-evacuated container 226 toward a piercing element 228, as shown by FIG. 12. Referring to FIG. 13, in another embodiment, an actuation device 230 includes a Y-port 232 that provides a fluid-receiving passageway 234 bypassing a pre-evacuated container 236. Referring to FIG. 14, as discussed above, bodily fluid is shown entering a pre-evacuated container 238 while bodily fluid 240 also bypasses the pre-evacuated container 238. In other embodiments, a one-way valve, represented by dashed lines 242 may be provided that prevents liquid from entering the pre-evacuated container 238.

The above-described fluid collection apparatuses may be suitable for pleural and peritoneal drainage operations and include an actuation device that houses a pre-evacuated container, such as a vial, therein. The pre-evacuated container houses a sealed partial vacuum that can be communicated through a drainage line that is in communication with an implanted conduit. The partial vacuum can be used to start draining bodily fluid from a body cavity or other location on the body and then gravity can be used to continue the drainage operation until the drainage is stopped in some way. A flow control device may be provided that allows for manual control of the drainage rate, which can improve patient comfort during the drainage operation.

Embodiments can be described with reference to the following numbered clauses, with certain features laid out in the dependent clauses:

Clause 1: A drainage system for draining bodily fluid, the drainage system comprising: a fluid-receiving body having a fluid-receiving chamber located therein that receives a bodily fluid through an inlet; and an actuation device comprising a housing with a container-receiving chamber that is sized and configured to receive a pre-evacuated container therein, the actuation device comprising an actuation member that, upon actuation, reduces a distance between a seal of the pre-evacuated container and a piercing element such that the piercing element pierces the seal of the pre-evacuated container providing communication of a negative pressure inside the pre-evacuated container to a fluid-receiving passageway to draw bodily fluid into the fluid-receiving passageway and toward the fluid-receiving body.

Clause 2: The drainage system of clause 1 further comprising a one-way valve located at the inlet of the fluid-receiving body that provides a single fluid flow direction into the fluid-receiving body.

Clause 3: The drainage system of clause 1 or 2, wherein the actuation device comprises the fluid-receiving passageway, the container-receiving chamber is liquidly isolated from the fluid-receiving passageway.

Clause 4: The drainage system of any of clauses 1 to 3, wherein the actuation member is movably connected to the housing and contacts the pre-evacuated container such that movement of the actuation member relative to the housing moves the pre-evacuated container toward the piercing element.

Clause 5: The drainage system of clause 4, wherein the actuation member comprises a button that, when moved relative to the housing, moves the pre-evacuated container toward the piercing element.

Clause 6: The drainage system of clause 5, wherein the actuation device comprises a clip between the button and the housing that inhibits movement of the button or slide relative to the housing.

Clause 7: The drainage system of any of clauses 1 to 6 further comprising a pre-evacuated container having a negative pressure therein and a seal that prevents air from entering the pre-evacuated container.

Clause 8: The drainage system of clause 7, wherein an end of the pre-evacuated container provides the actuation device.

Clause 9: The drainage system of any of clauses 1 to 8, wherein the housing has a fluid inlet that provides ingress of bodily fluid to the fluid-receiving passageway and a fluid outlet that provides egress of bodily fluid from the fluid-receiving passageway.

Clause 10: The drainage system of clause 9 further comprising a first drainage tube connected to the fluid inlet and a second drainage tube connected to the fluid outlet.

Clause 11: The drainage system of clause 10, wherein the fluid-receiving body is a bag that is in fluid communication with the second drainage tube.

Clause 12: A method of forming a drainage system for draining a bodily fluid, the method comprising: connecting a first drainage tube to an inlet of a housing of an actuation device, the actuation device comprising: the housing comprising a container-receiving chamber that is sized and configured to receive a pre-evacuated container therein, the actuation device comprising an actuation member that, upon actuation, reduces a distance between a seal of the pre-evacuated container and a piercing element such that the piercing element pierces the seal of the pre-evacuated container providing communication of a negative pressure inside the pre-evacuated container to a fluid-receiving passageway to draw bodily fluid into the fluid-receiving passageway and toward the fluid-receiving body; and placing a pre-evacuated container in the container receiving chamber.

Clause 13: The method of clause 12, wherein the actuation member is movably connected to the housing and contacts the pre-evacuated container such that moving the actuation member relative to the housing moves the pre-evacuated container toward the piercing element.

Clause 14: The method of clause 13, wherein the actuation member comprises a button such that moving the button moves the pre-evacuated container toward the piercing element.

Clause 15: The method of clause 14 comprising inhibiting movement of the button relative to the housing using a clip located between the button or slide and the housing.

Clause 16: The method of clause 14, wherein the button covers an opening to the container-receiving chamber through which a pre-evacuated container is received.

Clause 17: The method of any of clauses 12 to 16, wherein the pre-evacuated container has a seal that prevents air from entering the pre-evacuated container.

Clause 18: The method of any of clauses 12 to 17, wherein the housing has the fluid inlet that provides ingress of bodily fluid to the fluid-receiving passageway and a fluid outlet that provides egress of bodily fluid from the fluid-receiving passageway.

Clause 19: The method of clause 18 further comprising connecting a second drainage tube connected to the fluid outlet.

Clause 20: The method of clause 19 further comprising connecting the second drainage tube to a fluid-collecting bag.

Clause 21: The method of clause 12 further comprising replacing the pre-evacuated container in the container receiving chamber with another pre-evacuated container.

Clause 22: A drainage system for draining bodily fluid, the drainage system comprising: a fluid-receiving body having a fluid-receiving chamber located therein that receives a bodily fluid through an inlet; and an actuation device comprising a housing with a container-receiving chamber sized and configured to receive a pre-evacuated container therein, the actuation device comprising an actuation member that, upon actuation, reduces a distance between a seal of the pre-evacuated container and a piercing element such that the piercing element pierces the seal of the pre-evacuated container; wherein bodily fluid bypasses the pre-evacuated container through a fluid-receiving passageway and is received by the fluid-receiving body.

Clause 23: The drainage system of clause 22 further comprising a pre-evacuated container located in the container receiving chamber, the pre-evacuated container having a closed end moved by the actuation member.

Clause 24: The drainage system of clause 22, wherein the actuation member comprises a button that, when moved relative to the housing, moves the pre-evacuated container toward the piercing element.

Clause 25: The drainage system of clause 22, wherein the actuation device comprises the fluid-receiving passageway.

It will be apparent to those skilled in the art that various modifications and variations can be made to the embodiments described herein without departing from the spirit and scope of the claimed subject matter. Thus it is intended that the specification cover the modifications and variations of the various embodiments described herein provided such modification and variations come within the scope of the appended claims and their equivalents.

1. A drainage system for draining bodily fluid, the drainage system comprising:
a fluid-receiving body having a fluid-receiving chamber located therein that is configured to receive a bodily fluid; and
an actuation device comprising a housing with a container-receiving chamber that is sized and configured to receive a pre-evacuated container therein, the actuation device comprising an actuation member that, upon actuation, is configured to reduce a distance between a seal of the pre-evacuated container and a piercing element such that the piercing element pierces the seal of the pre-evacuated container inside the container-receiving chamber thereby providing communication of a negative pressure inside the pre-evacuated container to a fluid-receiving passageway to draw bodily fluid into the fluid-receiving passageway and toward the fluid-receiving body.

2. The drainage system of claim 1 further comprising a one-way valve located at an inlet of the fluid-receiving body that provides a single fluid flow direction into the fluid-receiving body.

3. The drainage system of claim 1, wherein the actuation device comprises the fluid-receiving passageway, the container-receiving chamber is liquidly isolated from the fluid-receiving passageway.

4. The drainage system of claim 1, wherein the actuation member is movably connected to the housing and contacts the pre-evacuated container such that movement of the actuation member relative to the housing moves the pre-evacuated container toward the piercing element.

5. The drainage system of claim 4, wherein the actuation member comprises a button that, when moved relative to the housing, moves the pre-evacuated container toward the piercing element.

6. The drainage system of claim 5, wherein the actuation device comprises a clip between the button and the housing that inhibits movement of the button or slide relative to the housing.

7. The drainage system of claim 1 further comprising a pre-evacuated container having a negative pressure therein and a seal that prevents air from entering the pre-evacuated container.

8. The drainage system of claim 7, wherein an end of the pre-evacuated container provides the actuation device.

9. The drainage system of claim 1, wherein the housing has a fluid inlet that provides ingress of bodily fluid to the fluid-receiving passageway and a fluid outlet that provides egress of bodily fluid from the fluid-receiving passageway.

10. The drainage system of claim 9 further comprising a first drainage tube connected to the fluid inlet and a second drainage tube connected to the fluid outlet.

11. The drainage system of claim 10, wherein the fluid-receiving body is a bag that is in fluid communication with the second drainage tube.

12. A method of forming a drainage system for draining a bodily fluid, the method comprising:
connecting a first drainage tube to an inlet of a housing of an actuation device, the actuation device comprising:
the housing comprising a container-receiving chamber that is sized and configured to receive a pre-evacuated container therein, the actuation device comprising an actuation member that, upon actuation, is configured to reduce a distance between a seal of the pre-evacuated container and a piercing element such that the piercing element pierces the seal of the pre-evacuated container inside the container-receiving chamber thereby providing communication of a negative pressure inside the pre-evacuated container to a fluid-receiving passageway to draw bodily fluid into the fluid-receiving passageway and toward a fluid-receiving body; and
placing a pre-evacuated container in the container receiving chamber.

13. The method of claim 12, wherein the actuation member is movably connected to the housing and contacts the pre-evacuated container such that moving the actuation member relative to the housing moves the pre-evacuated container toward the piercing element.

14. The method of claim 13, wherein the actuation member comprises a button such that moving the button moves the pre-evacuated container toward the piercing element.

15. The method of claim 14 comprising inhibiting movement of the button relative to the housing using a clip located between the button or slide and the housing.

16. The method of claim 14, wherein the button covers an opening to the container-receiving chamber through which a pre-evacuated container is received.

17. The method of claim 12, wherein the pre-evacuated container has a seal that prevents air from entering the pre-evacuated container.

18. The method of claim 12, wherein the housing has the fluid inlet that provides ingress of bodily fluid to the fluid-receiving passageway and a fluid outlet that provides egress of bodily fluid from the fluid-receiving passageway.

19. The method of claim 18 further comprising connecting a second drainage tube connected to the fluid outlet.

20. The method of claim 19 further comprising connecting the second drainage tube to a fluid-collecting bag.

21-25. (canceled)
